(19) United States
(12) Patent Application Publication
(10) Pub. No.: US 2019/0053936 A1
(43) Pub. Date: Feb. 21, 2019

(54) TISSUE RETENTION STRAP

(71) Applicant: MD Invent Solutions, Aiken, SC (US)

(72) Inventor: Jessica S. WHITTLE, Hixson, TN (US)

(21) Appl. No.: 16/102,920

(22) Filed: Aug. 14, 2018

Related U.S. Application Data
(60) Provisional application No. 62/546,654, filed on Aug. 17, 2017.

(51) Int. Cl. A61F 5/37; A61B 17/02

(52) U.S. Cl. CPC A61F 5/3776; A61B 2017/00951; A61B 2017/0212; A61B 17/02

(57) ABSTRACT

Disclosed herein are systems and methods of using a multi-functional tissue retention strap to displace and hold excess tissue out of the way of a target treatment area on a patient's body during a medical procedure. The tissue retention strap may be used to expose an area of the patient's body for a variety of purposes. The tissue retention straps of the present disclosure are ultralightweight and only require one person to operate.

### CROSS-REFERENCE TO RELATED APPLICATION(S)

The present patent application claims priority to U.S. Provisional Application No. 62/546,654, filed Aug. 17, 2017, and entitled “Tissue Retention Strap”, the entire disclosure of which is incorporated herein by reference.

### FIELD OF THE DISCLOSURE

The present disclosure relates generally to excess tissue displacement and retention for medical procedures and treatments. More specifically, the present disclosure relates to tissue retention straps that may be used to displace and hold excess tissue out of the way of a target treatment area on a patient's body, such as when inserting catheters and/or vascular devices into femoral arteries and/or veins, for example. The tissue retention strap may be used to expose an area of the patient's body for a variety of purposes, including wound care, cleaning or bathing, airing, etc.

### BACKGROUND OF THE DISCLOSURE

Often in emergency medical or even routine health care, areas of a patient's body (e.g., groin and other hard to reach regions) may need to be accessed for various reasons, such as wound care, abscess drainage, and the insertion of vascular devices. These body regions may be extremely difficult to access in certain patients, such as those who are obese or have excess tissue in the area (e.g., large overhanging panniculus).

One common procedure requiring access to the groin region on a patient is the insertion of a vascular device, such as the placement of a central venous line. Central venous lines are large intravenous catheters placed into large blood vessels for delivering medications (e.g., potentially caustic medicines, multiple drugs simultaneously, large volume delivery). Central venous lines may act as literal lifelines in critically ill patients—particularly when peripheral intravenous access is difficult or impossible to obtain.

Placing central venous lines may be most difficult in morbidly obese patients, especially if they are hypotensive with collapsible vessels, which can be hard to canalize even in the best circumstances. Morbid obesity correlates with increased distances from the skin's surface to blood vessels, causing further difficulty in peripheral vascular access since blood vessels and anatomical landmarks may be harder to identify. Moreover, the subcutaneous tissue of morbidly obese patients may move much more freely, thus even minimal/normal arm movements may easily dislodge a catheter from a blood vessel.

The placement of central venous lines in critically ill, morbidly obese patients is often necessary due to the lack of access to other blood vessels. However, inserting a central venous line in morbidly obese or bariatric patients may be challenging due to the inability to create adequate exposure of the relevant body regions as needed. Maneuvering the excess tissue away from the area to be exposed may require several people, in addition to the person performing the placement of the central venous line and/or other procedure. The amount of people and time required for assisting in these procedures adds significantly to healthcare costs. Additionally, from the patient's perspective, having more people touch or handle your body can add to the embarrassment and/or stress of any medical procedure at a time where being calm and comfortable may be critical physical and mental healing.

Some conventional solutions have included the use of tape to temporarily displace excess skin from a treatment area on a patient's body for a time, such as during surgical or other medical procedures. However, tape may fail, leading to disastrous results if sensitive instruments are suddenly moved violently or if external pollutants are introduced into an open incision or wound. Moreover, tape may be harsh on the skin, especially for burn patients. Additionally, removal of tape may irritate skin and/or painfully pull on or rip off skin and hair.

Other methods that hospitals have adopted for dealing with this issue of retaining excess tissue include utilizing sheets, gowns, etc. as makeshift slings. However, this method still requires the use of multiple people, and the fabric of the sling may not provide proper support or a reliable friction hold due to rolling or wrinkling of the fabric. Similarly, U.S. Patent Publication No. 2005/0150503 discloses an abdominal support sling “for repositioning an apron of abdominal fat tissue”, requiring a bulk of fabric for the sling portion, which is susceptible to the same issues and is not very adaptable to different types or shapes of excess tissue.

Additionally, U.S. Pat. Nos. 6,846,220, 7,455,649, 7,938,121, and 8,764,691, as well as U.S. Patent Publication No. 2012/0024296 disclose support garments and slings that wrap around a patient's body, utilizing the back or shoulders for leverage. These prior solutions all require having to place or wrap the apparatus around the patient, which may be quite difficult and requires extra time.

It is now estimated that approximately 35% of the US population is obese and almost 7% are considered extremely obese. These patients are more likely to have numerous medical co-morbidities and to be older, which also means they are more likely to become critically ill. As these rates are expected to continue to rise, this represents a large proportion of patients and intravascular access is a critical healthcare problem.

Thus, what is still needed in the art is a process for easily displacing excess tissue on a patient to expose relevant areas for enough time to perform needed treatments and/or procedures that is comfortable for the patient, efficient, reliable, and does not require several people.

### BRIEF SUMMARY OF THE DISCLOSURE

The present disclosure addresses the issues associated with accessing patient body regions by using a strap or similar device to retain excess tissues away from relevant treatment areas during medical procedures and/or treatments.

### BRIEF DESCRIPTION OF THE DRAWINGS

The present invention is illustrated and described herein with reference to the drawings, in which:

FIG. 1 is a schematic diagram of an example embodiment of a tissue retention strap for retaining excess tissue away from a treatment region on a patient's body, in accordance with the present disclosure;

FIG. 2 is an illustration of an example patient with excess tissue, blocking a treatment region, to be addressed by a tissue retention strap, in accordance with the present disclosure;

FIG. 3 is an illustration of the application of an example embodiment of a tissue retention strap to the excess tissue of the patient, in accordance with the present disclosure;

FIG. 4 is an illustration of the attachment of an anchor strap on the tissue retention strap of FIG. 3 to the patient's bed, in accordance with the present disclosure;

FIG. 5 is an illustration of the tissue retention strap of FIG. 3 allowing access to the desired treatment region of the patient's body, in accordance with the present disclosure;

FIG. 6 is an illustration of tightening an anchor strap on the tissue retention strap of FIG. 3 using a built-in adhesive strip, in accordance with the present disclosure;

FIG. 7 is an illustration of the tissue retention strap of FIG. 3 fully installed on the patient, allowing for access to the treatment region, in accordance with the present disclosure; and

FIG. 8 is an illustration of example fastening ends for a tissue retention strap, in accordance with the present disclosure.

### DETAILED DESCRIPTION OF THE DISCLOSURE

Disclosed herein are systems and methods for shifting excess tissue on a patient to access and/or expose a treatment region on the patient's body using a tissue displacement or retention strap. The tissue retention strap is thin, flexible, and easy to implement for a patient without requiring help from others. The excess tissue retention strap may be made from ultralightweight medical-grade materials.

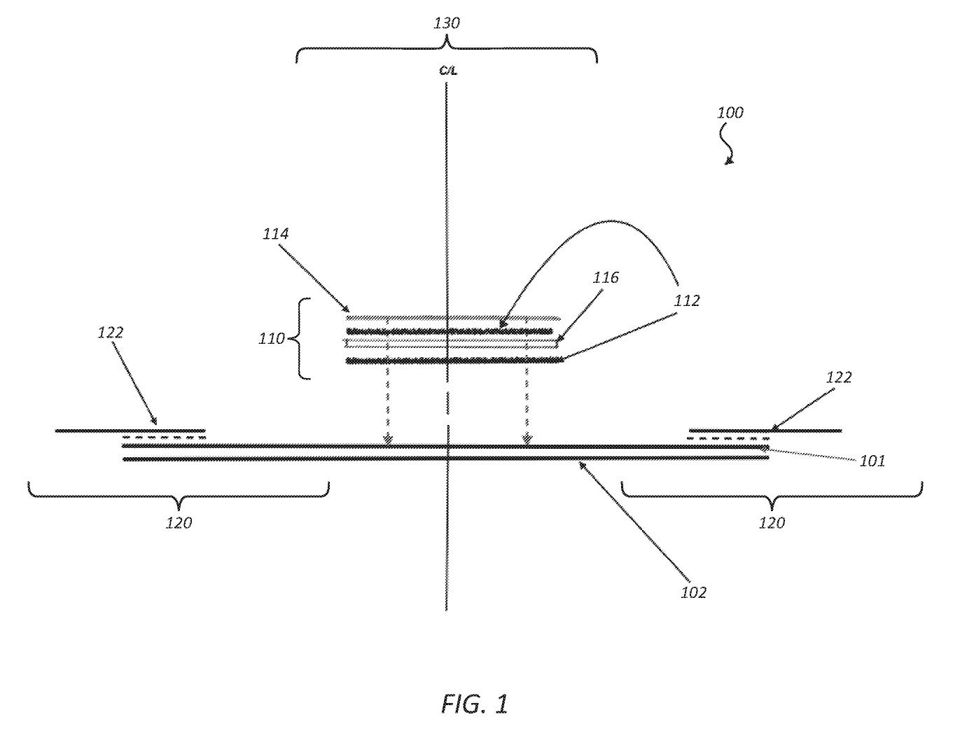

As shown in FIG. 1, a tissue retention strap 100 may be an ultralightweight, thin strip for retaining excess tissue away from a treatment region on a patient's body. The tissue retention strap 100 may be made out of medical-grade materials, such as woven fabric medical tape, for example. Using medical-grade materials not only may provide a safer experience for the patient, but also may allow the tissue retention strap 100 to, optionally, be sterilized and re-used. This is advantageous for lowering healthcare costs.

The tissue retention strap 100 may be made from two main layers spanning its entire length (or at least a substantial portion thereof)—specifically, (1) an inner layer 101, facing the patient, and (2) and outer layer 102. The inner layer 101 may be laminated, glued, or otherwise affixed to the outer layer 102 along the majority of the length of the tissue retention strap 100.

The inner layer 101 may include an adhesive tape layer, comprising an adhesive layer atop a backing or substrate layer. The adhesive within the adhesive tape layer may be a hypoallergenic acrylate formulation. The substrate within the adhesive tape layer may be a non-stretching, woven fabric, such as rayon acetate (e.g., 3M™ 1538 Rayon Acetate Woven Fabric Medical Tape). The woven, non-stretch fabric of the inner layer 101 may advantageously add to the strength, reliability, and control of the tissue retention strap 100.

The outer layer 102 of the tissue retention strap 100, which faces away from the patient, may include fasteners along at least some portions of its length. For example, the entire outer layer 102 may include loop fasteners covering its surface. The loop fastener material of the outer layer 102, in some embodiments, may be a low-profile closure surface made of nylon and polyethylene (e.g., 3M™ 7330 Low Profile Loop Fastener Roll with No Adhesive).

The length of the tissue retention strap 100 may be optimized for wrapping around the patient and reaching various nearby anchors for attachment, without being too lengthy as to get in the way of the medical procedure and/or treatment process. For example, the tissue retention strap 100 may be approximately 6 ft. from end to end. Additionally, the width of the tissue retention strap 100 may fall within the range from about 1.5 in. to about 12 in., such that the tissue retention strap 100 is not so narrow that it might exert too high a pressure on the excess tissue of the patient, and not so wide as to be susceptible to wrinkles that would cause discomfort against the skin of the patient. For example, the width of the tissue retention strap 100 may be approximately 2 in. and provide adequate support and comfort for the patient.

In addition to the dual layers (i.e., inner layer 101 and outer layer 102) stacked along the length of the tissue retention strap 100, the tissue retention strap 100 may be divided up into adjacent lateral regions, consisting of two main region types: (1) an anchor region 120 located along each of the two ends of the tissue retention strap 100, and (2) a main contact region 130 in the middle of the tissue retention strap 100 between the two anchor regions 120 of the strap ends.

The anchor regions 120 of the tissue retention strap 100 include the free ends and fasteners of the tissue retention strap 100 for anchoring the tissue retention strap 100 to points or anchors surrounding—and fixed relative to—the patient. For example, anchors may be provided by rails or other bracing structures on a bed and/or hospital gurney. Additionally or alternatively, secure mounting brackets and/or other sturdy fixtures within a room may also act as anchors for sustaining large applied loads.

The main contact region 130 is the remaining midsection of the tissue retention strap 100 not already included in either anchor region 120. The main contact region 130 of the tissue retention strap 100 is the portion that comes into contact with the excess tissue of the patient, directly or indirectly, to provide mobility and support to the patient's body with minimal discomfort. The main contact region 130 of the tissue retention strap 100 may include an adhesive stack 110 for providing a sticky pad area facing the patient for attaching the tissue retention strap 100 directly to the patient's skin. The adhesive stack 110 may attach itself directly to the inner layer 101 of the tissue retention strap 100 using an adhesive layer 112. The adhesive layer 112 may include polyethylene double-sided medical tape, consisting of two adhesive layers surrounding a carrier layer. For example, the adhesive layers 112 may include hypoallergenic acrylate layers with polyethylene film in between (e.g., 3M™ 9889 Polyethylene Double-Sided Medical Tape). Alternatively, the adhesive layer 112 may be a simple layer of glue.

The rest of the adhesive stack 110 may comprise a contact adhesive layer 114 and a substrate layer 116 with another adhesive layer 112 in between. The contact adhesive layer 114 may include a backing layer (e.g., heat sealable polyethylene film), an adhesive layer (e.g., absorbent tackified hydrocolloid or other medical-grade adhesive), and a protective liner for covering the hydrocolloid adhesive layer until it is ready to be attached to the patient. As a non-limiting example, the contact adhesive layer 114 may be made with a backing layer with a thickness of about 1.7 mil, a hydrocolloid adhesive layer with a thickness of about 27.8 mil, and an 80# two-sided silicone liner (e.g., 3M™ 9945) Hydrocolloid Adhesive Medical Tape with Heat-Sealable Polyethylene Backing). Use of the hydrocolloid adhesive rather than conventional tape advantageously allows the tissue retention strap 100 to be much gentler on patient skin. This could open up more possible procedures for patients with burns or other characteristics causing their skin to be more fragile and/or have increased sensitivities.

Further, the substrate layer 116 of the adhesive stack 110 may be a polyethylene terephthalate (PET) material, laminated and/or glued to the contact adhesive layer 114 via the adhesive layer 112, as described above. In some embodiments, the substrate layer 116 may have a thickness between about 0.010 in. to about 0.020 in. The substrate layer 116 advantageously adds thickness to the adhesive stack 110 of the main contact region 130 in the center of the tissue retention strap 100. This added thickness increases the rigidity of the sticky pad area in the contact adhesive layer 114. Thus, despite the relatively thin width of the tissue retention strap 100 (e.g., about 2 in.), rather than the fabric and/or tape layers 101/102 wrinkling or collapsing back on themselves at the attachment point to the patient's body, the substrate layer 116 allows the tissue retention strap 100 to maintain its shape and/or form even when tension is applied to and propagates through the strap 100. By preserving its relatively more rigid shape, the main contact region 130 of the tissue retention strap 100 may beneficially support the excess tissue of the patient in a more comfortable manner, since the pressure forces from the tension in the tissue retention strap 100 may be distributed over a larger surface area (i.e., the contact adhesive layer 114 in the adhesive stack 110 of the main contact region 130 on the tissue retention strap 100). Moreover, because the rigidity within the main contact region 130 maintains the rectangular shape of the adhesive stack 110 and resists deformation by the counteracting forces of the excess tissues, the relevant treatment region may achieve the maximum accessibility and/or exposure possible. In particular, without the added rigidity of the substrate layer 116 within the adhesive stack 110, the excess tissue of the patient may tend to roll or otherwise curl the edges of the tissue retention strap 100 and thereby envelop the strap turned thin cord, having allowed the panniculus or other surplus flesh to spill over into the area, from which the tissue retention strap 100 aimed to relocate the very same.

To ensure proper bonding, this sticky pad area/adhesive stack 110 of the main contact region 130 may be approximately about 2 in. in width (i.e., spanning most if not all of the width of the tissue retention strap 100) and about 6 in. in length. If the surface area of the contact adhesive layer 114 in the adhesive stack 110 is too large, the tissue retention strap 100 may start to become more difficult to remove.

The anchors surrounding the patient may allow for any applied pulling and/or tension forces, directed back toward the treatment region, to be easily transferred through the tissue retention strap 100 and around the anchors, thus converting them into pushing forces that act to move excess tissue away from the treatment region. Once the tissue retention strap 100 has displaced the excess tissue enough to provide adequate access and/or exposure to the relevant treatment area, the anchor regions 120 may be fastened around the anchors, so as to lock everything into position for the duration of the medical procedure and/or treatment process.

There are many ways in which the anchor regions 120 of the tissue retention strap 100 may be fastened to and/or around the anchors. In some embodiments, where the outer layer 102 of the tissue retention strap 100 is covered in loop fasteners, fastening regions 122 may be attached to the tissue retention strap 100 and made of complementary hook fasteners (e.g., 3M™ 7336 Low Profile Hook Fastener Roll with Adhesive). The end fastening regions 122 may be made from polyolefin hook fastener tape and adhered to the tissue retention strap 100 using a high tack medical adhesive, such as a synthetic rubber adhesive, for example. In some embodiments, there may be multiple end fastening regions 122 (e.g., 6 regions) attached to the tissue retention strap 100, thereby providing excellent shear strength for the tissue retention strap 100 system that resists any accidental release of the anchor regions 120. These end fastening regions 122 may be sized and distributed to provide the optimal hold while maintaining adjustability. For example, the end fastening regions 122 may be approximately 2 in. in width—in accordance with the overall uniform width of the tissue retention strap 100—and between about 2 in. and about 4 in. in length. Characteristics of the complementary fastening structures (e.g., fastener shape, flexibility, density, material) as well as the length and width dimensions of the end fastening regions 122 may be varied in order to optimize (not maximize) the strength of the hold. In particular, too high a strength may become uncomfortably restrictive. Therefore, the fastener can be tailored to give way at a certain maximum tension or shear force to ensure the patient is not harmed.

In some embodiments, the tissue retention strap 100 may include an adjustment mechanism, such as a ratcheting mechanism or friction hold structure. The adjustment mechanism may allow the straps of the tissue retention strap 100 to be quickly and easily tightened after the main contact pad of the tissue retention strap 100 is placed on the patient 150 and the strap ends are anchored. The adjustment mechanism may include a lock or other similar device so that the tissue retention strap 100 is not suddenly released accidentally.

In some embodiments, the tissue retention strap 100 may be made from a stretching and/or elastic material (e.g., rubber). For example, wide latex bands (such as resistance bands used for exercise and/or physical therapy) may be used for retaining excess tissue away from relevant body regions to be exposed. The latex may be medical-grade. Rather than using the hook and loop fastener type as described above, the ends of the latex bands may be quickly and easily tied to fixed anchors around the patient 150. The latex bands may additionally include adhesive on one side of the middle of the tissue retention strap 100 for better tissue retention through maintaining optimal positioning. The adhesive may be non-irritating for the patient's skin. Additionally, the adhesive may be resistant to sticking to hair, so that removal of the tissue retention strap 100 may be relatively painless.

In some embodiments, the tissue retention strap 100 may be modular. Modular tissue retention straps may include a main contact pad and add-on straps. The main contact pad of the tissue retention strap 100 may be placed adjacent the patient's skin. The main contact pad may include adhesive for better tissue retention and easier placement. The adhesive may be non-irritating for the patient's skin. Additionally, the adhesive may be resistant to sticking to hair, so that removal of the tissue retention strap 100 may be relatively painless. The main contact pad may include loops and/or other fasteners (e.g., velcro, buckles, snaps, eyes, eyelets, notches) for connecting the add-on straps. The add-on straps may include hooks and/or other fasteners complementary to the main contact pad fasteners for connecting to the main contact pad. The add-on straps may be of standard or various lengths. At least one end of the add-on strap may include a fastener-type compatible with the fastener-type of other add-on straps. The add-on straps may include anchor straps with fasteners compatible with fixed anchors surrounding the patient 150 (e.g., rails of a bed or hospital cart). Alternatively, the anchor straps may be integrated into a hospital bed, gurney, stretcher, or other fixed anchors surrounding a patient 150.

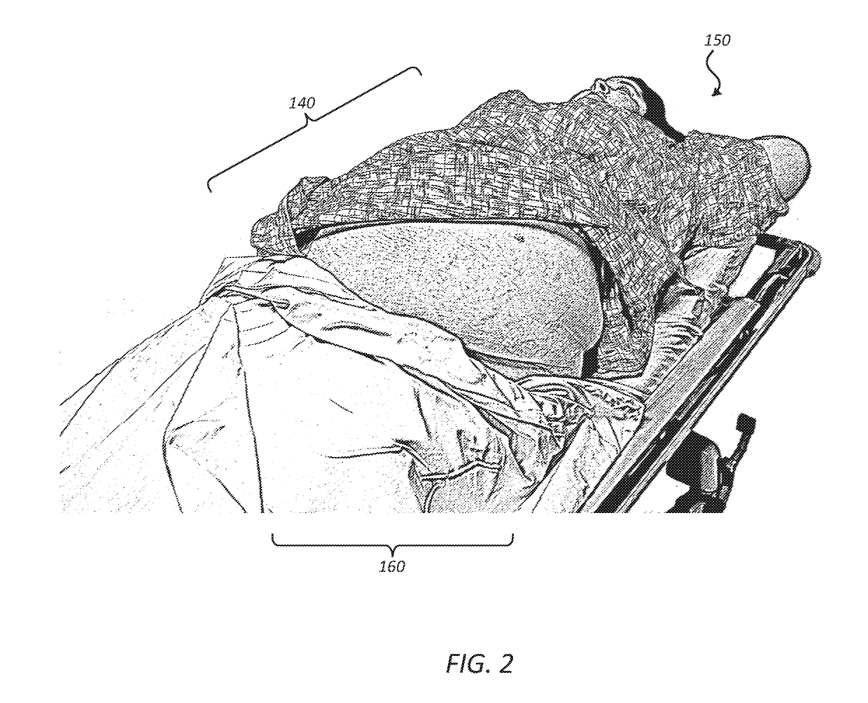

FIG. 2 shows an example of a patient 150 with excess tissue 140, blocking a treatment region 160. This excess tissue problem may be solved by performing the steps of the following method to implement and utilize the tissue retention strap 100, described above.

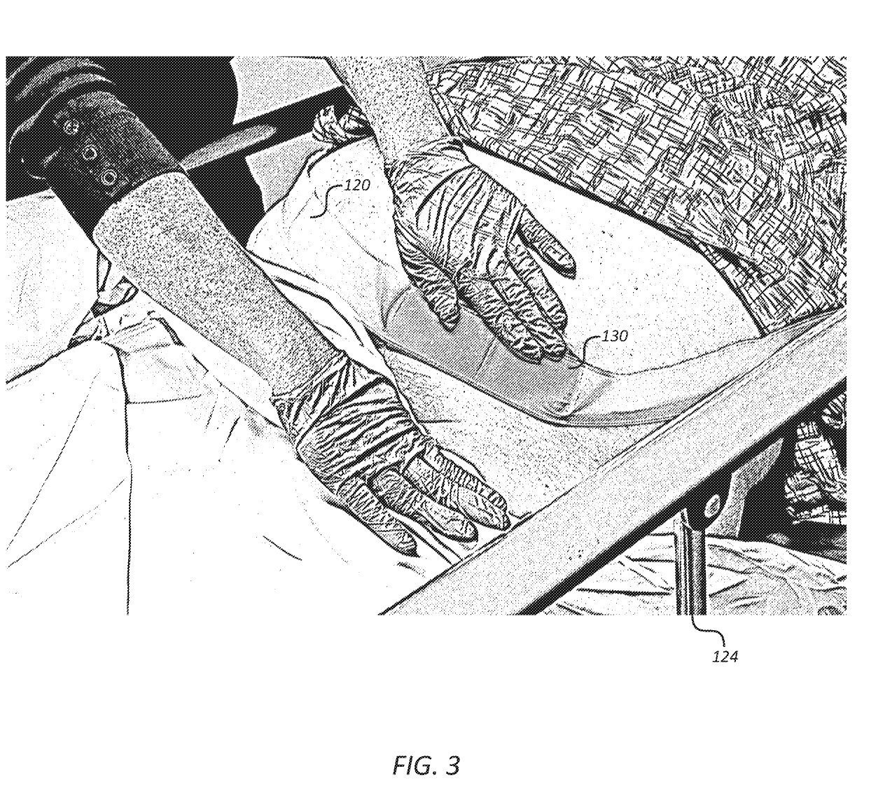

As shown in FIG. 3, the excess tissue 140 of the patient 150 may be slightly maneuvered away by hand from the relevant treatment region 160 to reveal a skin surface area within the excess tissue 140, to which the tissue retention strap 100 may be applied. This skin surface area may need to be cleaned before attachment can be made. To attach the tissue retention strap 100 to the patient 150, the liner of the contact adhesive layer 114 may be removed. The main contact region 130—and more specifically, the adhesive stack 110 with the contact adhesive layer 114—of the tissue retention strap 100 may then be pressed against the excess tissue 140 of the patient 150. The hydrocolloid adhesive of the contact adhesive layer 114 may then adhere to the excess tissue 140 of the patient 150. Once the main contact region 130 is positioned and/or locked into place, the anchor regions 120 of the tissue retention strap 100 are free to be anchored and adjusted. Additionally or alternatively, one or more of the anchor regions 120 of the tissue retention strap 100 may be fastened to the anchors before the main contact region 130 is applied to the excess tissue 140 of the patient 150.

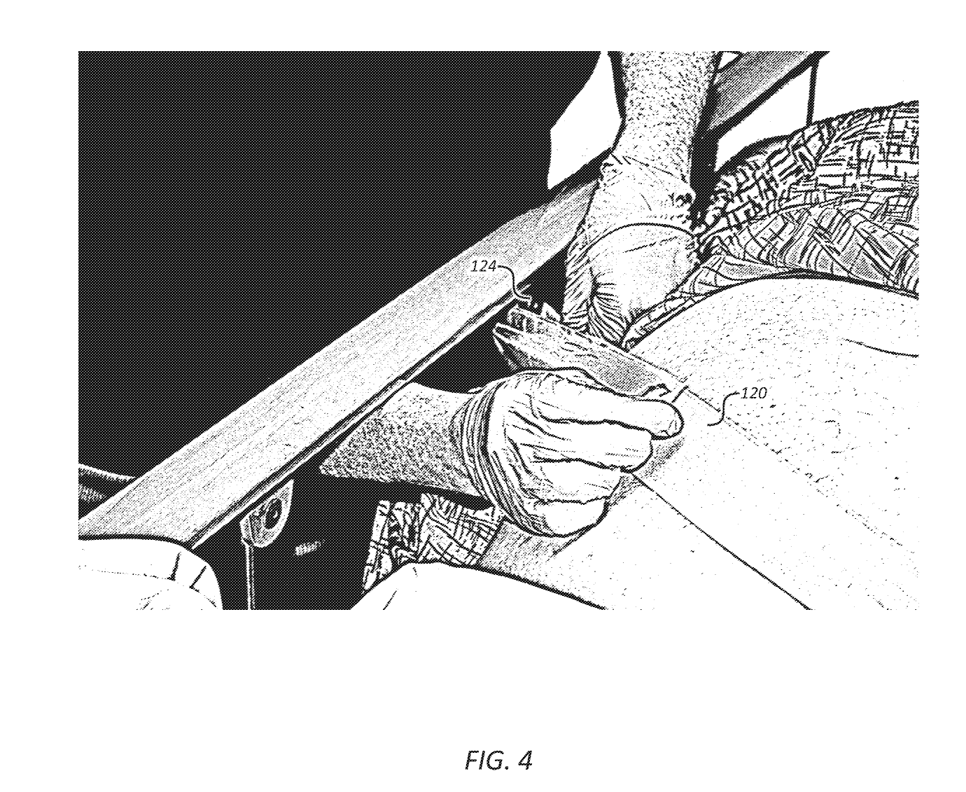

As shown in FIG. 4, the anchor regions 120 of the tissue retention strap 100 of FIG. 3 may be attached to the patient's side bed rails or any other suitable anchor 124. In some embodiments, the anchor regions 120 include both loop fastening structures along the outer layer 102 of the tissue retention strap 100 and end fastening regions 122, featuring complementary hook fastening structures that stick to the outer layer 102. Thus, the end anchor regions 120 of the tissue retention strap 100 may each be easily looped around an anchor 124 and fastened to themselves, thereby providing a reliable hold for the excess tissue 140 of the patient 150. There are many other ways in which to fasten the anchor region 120 of the tissue retention strap 100 to itself or to an anchor, as discussed above.

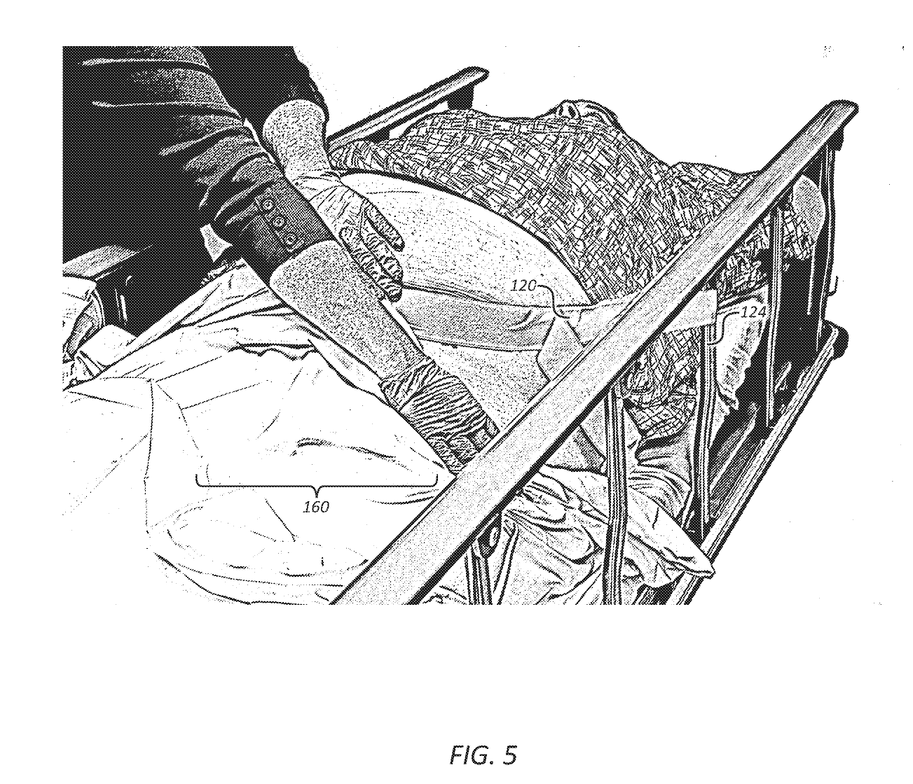

FIG. 5 shows how the implementation of the tissue retention strap 100 of FIG. 3 for the patient 150 allows access to the desired treatment region 160 via tightening and/or adjusting the anchor regions 120. While adjusting the tension in the tissue retention strap 100 with the end anchor regions 120, the excess tissue 140 of the patient 150 may need to be further maneuvered out of the way of the straps.

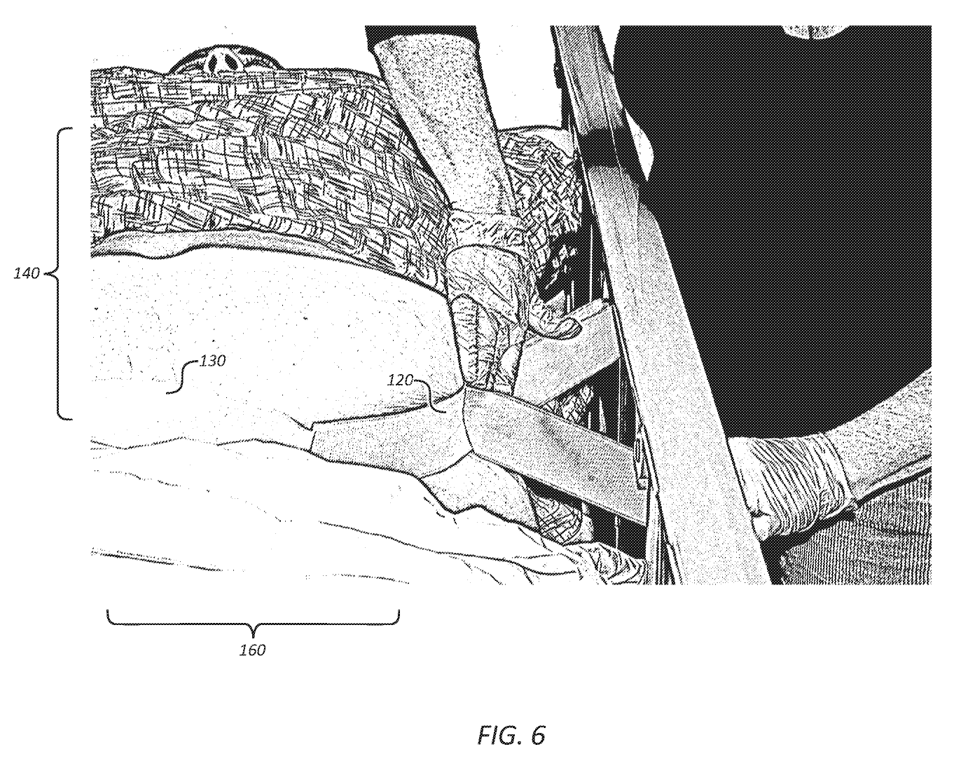

FIG. 6 shows how further adjustments may be made to the excess tissue 140 displacement by using the anchor region 120 to tighten the tissue retention strap 100 of FIG. 3 using the built-in fastener strips of the end fastening regions 122 to reveal more of the relevant treatment area 160. Moreover, FIG. 6 illustrates how the contact adhesive layer 114 of the main contact region 130 maintains a reliable bond with the excess tissue 140 of the patient 150 even under high tensions.

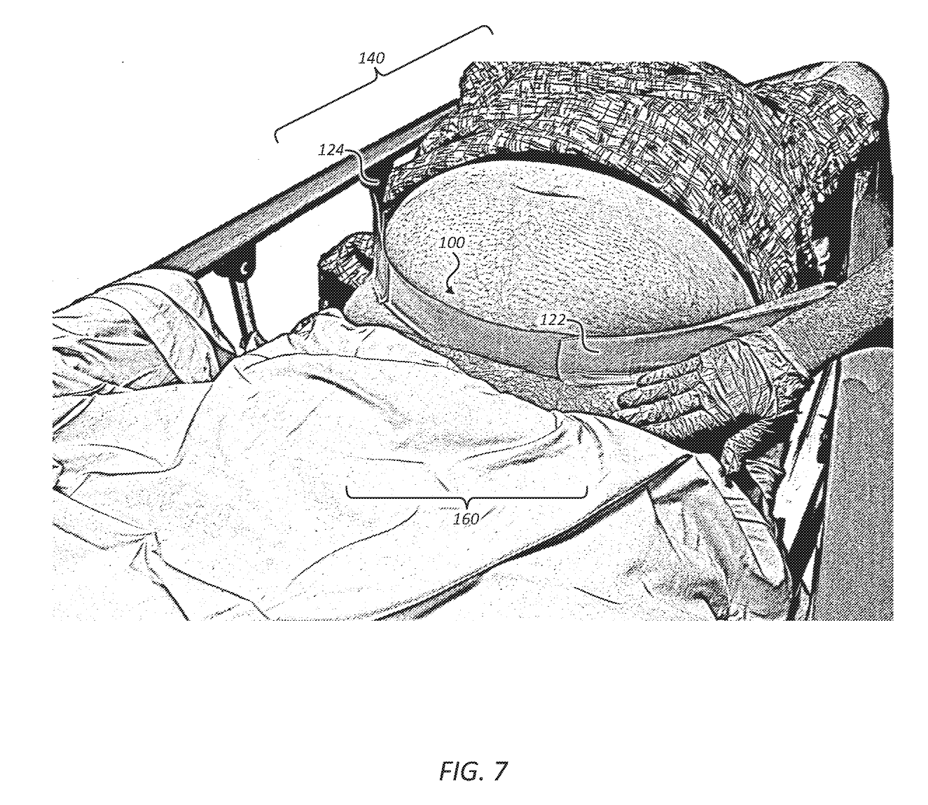

As shown in FIG. 7, the tissue retention strap 100 may be fully installed on the patient 150, allowing for greater access to the treatment region 160 for more invasive or involved medical procedures, with minimal discomfort for the patient 150. After the medical procedure and/or treatment is complete, the tissue retention strap 100 may be easily and instantly released by pulling on the end fastening regions 122. After use, the tissue retention strap 100 may be disposed of, recycled, or, optionally, sterilized and re-used.

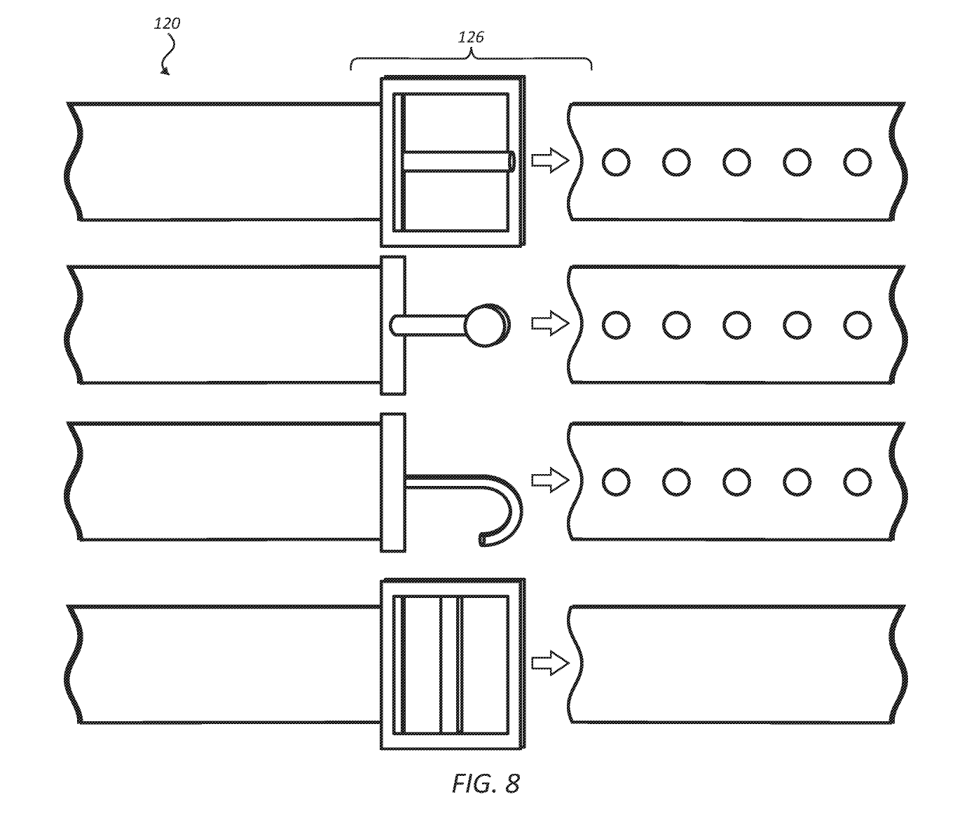

FIG. 8 shows example end anchor regions 120 with various fastener mechanisms 126, including a buckle, hole and post, hook and eye, and friction hold. Other fastener mechanisms 126 are possible, including a D-ring, a snap, a frog, a threaded clasp, and a magnetic clasp, for example.

Advantageously, some embodiments of the tissue retention strap 100 may allow just one person—even a patient 150 themselves—to be able to displace and retain any excess tissue away from a relevant area on the patient's body. This may be greatly helpful in in-home care environments where there is only one nurse or caregiver that otherwise would have to sacrifice the use of one hand in maneuvering tissue and/or request outside help, which may be difficult to obtain. Additionally, use of the tissue retention strap 100 by the patient 150 themselves may be tremendously beneficial in wound healing. For example, allowing the patient 150 to strap excess tissue back away from a body region with a wound may cause the wound to heal faster due to the air exposure and/or removal of friction or contact contaminants (e.g., skin-born bacteria, sweat).

Healthcare professionals and others may use the tissue retention strap 100 in a variety of environments (e.g., emergency medical departments, hospitals, doctors' offices, assisted and nursing care facilities, at home) to expose an area of a patient's body for a variety of purposes, including wound care, bathing, and airing out to speed healing of rashes or wounds. The tissue retention strap 100 may be used in hospital or emergency room environments for inserting catheters or vascular devices in the femoral artery or vein, and in particular, is used to assist in the placement of a central venous line (CVL). The tissue retention strap 100 may also provide access to areas for bathing patients in any setting. The tissue retention strap 100 may be used while a patient 150 is sleeping and/or resting. In some embodiments, multiple tissue retention straps 100 may be used at once.

The tissue retention strap 100 may be utilized for any region of the body. In some embodiments, the tissue retention strap 100 may aid in exposing the lateral area of the neck of a patient 150, in order to gain vascular access, for example. In some embodiments, the tissue retention strap 100 may lift pendulous breast tissue for the purposes of wound care, abscess drainage, suturing, and/or other medical needs. In some embodiments, the tissue retention strap 100 may help expose the lower abdomen for such procedures as performing a cesarean section, caring for Pfannenstiel incision wounds including, but not limited to, draining seromas removing staples or sutures, and cleaning, for example. The tissue retention strap 100 may be used to displace and hold thigh tissue for gynecologic examinations and procedures. In some embodiments, the tissue retention strap 100 may be used to gain better access to the groin or alternate intravascular access sites on burn patients, on whom tape may not be used due to skin fragility.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are contemplated thereby.

What is claimed is:

1. A tissue retention strap comprising:
a strip of loop fastener material with a central contact region in the middle of the strip; and
hook fastener material regions attached near ends of the strip, wherein the hook fastener material regions engage with the loop fastener material of the strip.

2. The tissue retention strap of claim 1, wherein the strip has a width between about 1.5 in. and about 12 in.

3. The tissue retention strap of claim 2, wherein the width is between about 2 in. and about 4 in.

4. The tissue retention strap of claim 3, wherein the width is about 2 in.

5. The tissue retention strap of claim 1, wherein at least a portion of the central contact region has a higher rigidity than the ends of the strip.

6. The tissue retention strap of claim 1, wherein the central contact region includes an adhesive pad.

7. The tissue retention strap of claim 6, wherein the adhesive pad is thicker than the ends of the strip.

8. The tissue retention strap of claim 1, further comprising a layer of non-stretch woven fabric attached to the strip of loop fastener material.

9. The tissue retention strap of claim 8, wherein the central contract region is located on the layer of non-stretch woven fabric.

10. The tissue retention strap of claim 1, wherein materials are each medical-grade.

11. A method retaining excess tissue of a patient away from a treatment area, the method comprising:
placing on the excess tissue a tissue retention strap comprising:
an excess tissue contact region in the middle of the strap, and
fastening regions along the ends of the strap;
doubling each of the ends of the strap around anchors positioned on opposite sides of the patient; and
engaging the doubled-over fastening regions with each other.

12. The method of claim 11, further comprising:
disengaging at least one fastening region;
repositioning the end of the strap near the disengaged fastening region closer to the excess tissue contact region, thereby causing the excess tissue contact region to move the excess tissue away from the treatment area; and
re-engaging the doubled-over fastening regions with each other.

13. The method of claim 11, wherein placing the tissue retention strap on the excess tissue includes affixing an adhesive pad in the excess tissue contact region to the excess tissue.

14. The method of claim 11, wherein the excess tissue contact region is thicker than the fastening regions.

15. A method of retaining excess tissues of a patient to expose a treatment area, the method comprising:
affixing an adhesive midsection of a tissue retention strap to the excess tissue of the patient; and
fastening ends of the tissue retention strap to anchors.

16. The method of claim 15, further comprising tightening the ends of the tissue retention strap around the anchors.

17. The method of claim 15, wherein the adhesive midsection is thicker than the rest of the tissue retention strap.

18. The method of claim 15, wherein the adhesive midsection includes a medical adhesive.

19. The method of claim 18, wherein the medical adhesive is a hydrocolloid adhesive.

20. The method of claim 15, wherein fastening the ends of the tissue retention strap to anchors includes engaging hook and loop structures along the ends of the tissue retention strap with each other.

21. A tissue displacement strap for retaining excess tissue of a patient comprising:
at least one adhesive portion near the middle of the tissue displacement strap for placement on the excess tissue of the patient; and
anchorable ends with a fastener mechanism for fastening and tightening the tissue displacement strap.

22. The tissue displacement strap of claim 21, wherein the fastener mechanism is at least one of a buckle, a hole and post, a hook and eye, a D-ring, a friction hold, a snap, a frog, a threaded clasp, and a magnetic clasp.
